(19) United States
(12) Patent Application Publication
(10) Pub. No.: US 2004/0242945 A1
(43) Pub. Date: Dec. 2, 2004

(54) Dehydrogenation of alkyl aromatic compound over a gallium-zinc catalyst

(76) Inventors: Joseph E. Pelati, Charleston, WV (US); Robert J. Gulotty JR., Midland, MI (US)

Correspondence Address: THE DOW CHEMICAL COMPANY, INTELLECTUAL PROPERTY SECTION, P. O. BOX 1967, MIDLAND, MI, 48641-1967, US

(21) Appl. No.: 10/853,372

(22) Filed: May 25, 2004

Related U.S. Application Data
(60) Provisional application No. 60/474,123, filed on May 29, 2003.

(51) Int. Cl. C07C 4/06

(52) U.S. Cl. 585/444

(57) ABSTRACT

A process for the dehydrogenation of an alkyl aromatic compound, preferably ethylbenzene, to a vinyl aromatic compound, preferably styrene, involving dehydrogenating the alkyl aromatic compound over a catalyst containing gallium, zinc, optionally alkali or alkaline earth, optionally manganese, and optionally a noble metal, deposited on a catalyst support, preferably, a transitional alumina. Optionally, the dehydrogenation feedstream may contain an alkane, preferably ethane, which is simultaneously dehydrogenated to an alkene, preferably ethylene. The dehydrogenation process can be integrated into a process of producing a vinyl aromatic compound, such as styrene, from a raw material base comprised of an alkane and an aromatic compound, such as, ethane and benzene.

### CROSS-REFERENCE TO RELATED APPLICATION

This application claims the benefit of U.S. Provisional Patent Application Serial No. 60/474,123, filed May 29, 2003.

### BACKGROUND OF THE INVENTION

In one aspect, this invention pertains to a novel process of dehydrogenating an alkyl aromatic compound, such as ethylbenzene, to form a vinyl aromatic compound, such as styrene. In a second aspect, this invention pertains to a novel process of dehydrogenating a feedstream containing an alkyl aromatic compound and an alkane to form a product stream containing a vinyl aromatic compound and an alkene, respectively. In this second aspect, the invention can be integrated into a larger process of preparing a vinyl aromatic compound using as raw materials an aromatic compound and an alkane. In a third aspect, this invention pertains to a novel catalyst composition containing gallium and zinc on a catalyst support.

The dehydrogenation of alkyl aromatic compounds, for example, ethylbenzene, isopropylbenzene, diethylbenzene, or p-ethyltoluene, finds utility in the preparation of styrene and substituted derivatives of styrene including α-methylstyrene, divinylbenzene, and p-methylstyrene. Styrene and its substituted derivatives are useful as monomers in the formation of polystyrenes, styrene-butadiene rubbers (SBR), acrylonitrile-butadiene-styrene (ABS), styrene-acrylonitrile (SAN), and unsaturated polyester resins. The dehydrogenation of alkanes, such as ethane, find utility in the preparation of alkenes, such as ethylene. Alkenes have well-known utility as monomers in the formation of poly(olefin) polymers and as reactants in various organic processes. Notably, alkenes can be used to alkylate aromatic compounds, such as benzene, to alkylated aromatic compounds, such as, ethylbenzene.

The primary manufacturing route to vinyl aromatic compounds, including styrene, involves the direct catalytic dehydrogenation of alkyl aromatic compounds, such as ethylbenzene. Patents representative of such a process include, for example, U.S. Pat. No. 4,404,123, U.S. Pat. No. 5,171,914, U.S. Pat. No. 5,510,552, and U.S. Pat. No. 5,679,878. The catalyst typically comprises iron oxide and, additionally, may comprise chromium oxide and potassium compounds as promoters. Since the process is highly endothermic, energy for the process is obtained by introducing superheated steam into the process reactor. Steam also functions to promote catalyst regeneration in situ during the dehydrogenation process. Usually, a high steam to ethylbenzene weight ratio is required, typically from greater than about 0.9/1 to about 2.0/1 and possibly higher, which disadvantageously imposes on the process a high energy input and a large water recycle.

Other art, represented by EP-A1-0,335,130, discloses the oxidative dehydrogenation of ethylbenzene in the presence of oxygen and a mixed oxide catalyst to form styrene. The mixed oxide may be represented by the formula:

xA.yB.zC.qO

wherein, for example, A is an alkali metal; B is selected from scandium, yttrium, lanthanum, actinium, aluminum, boron, and mixtures thereof; and C is selected from beryllium, magnesium, calcium, strontium, barium, radium, zinc, cadmium, mercury, and mixtures thereof. Oxidative dehydrogenation processes require the undesirable combination of oxygen and hydrocarbon feed. Moreover, the process disadvantageously produces large amounts of cracking and oxidation by-products.

Other art, represented by U.S. Pat. No. 5,430,211, discloses the dehydrogenation of ethane to ethylene over a catalyst containing gallium, zinc, or a platinum group metal, or combinations thereof, deposited on an aluminosilicate of mordenite structure. This reference is silent with respect to the dehydrogenation of alkyl aromatic compounds.

Yet other art, such as EP-B1-0,637,578 (SnamProgetti S.p.A.), discloses dehydrogenating a light paraffin, such as propane, over a catalyst comprising gallium, platinum, and one or more alkaline or alkaline earth metals, supported on an alumina support to yield light olefins, such as propylene. This reference is also silent with respect to the dehydrogenation of alkyl aromatic compounds over gallium catalysts.

With respect to integrated processes, EP-A1-0905112 (SnamProgetti S.p.A.) discloses a process for producing styrene comprising (a) feeding to an alkylation unit a stream containing benzene and ethylene; (b) mixing the stream at the outlet of the alkylation unit, containing ethylbenzene, with a stream consisting of ethane; (c) feeding the mixture thus obtained to a dehydrogenation unit containing a catalyst capable of contemporaneously dehydrogenating ethane and ethylbenzene; (d) feeding the product leaving the dehydrogenation unit to a separation section to produce a stream consisting of styrene and a stream containing ethylene; and (e) recycling the stream containing ethylene to the alkylation unit. As a dehydrogenation catalyst, it is taught to employ gallium oxide and platinum on alumina. Disadvantageously, the selectivity to styrene achieved with this process is lower than desired, as is the ethane conversion. The reference is also silent with regard to zinc.

Y. Okimura, et al. discloses in Catalysis Letters, 52 (1998) 157-161, a “Zn—Al—Ga complex oxide” having a spinel structure, as determined by X-ray analysis. The complex oxide is disclosed to be used in the catalytic reduction of nitrogen oxides.

In view of the above, a need exists for an improved dehydrogenation process to convert an alkyl aromatic compound, such as ethylbenzene, to a vinyl aromatic compound, such as styrene. It would be desirable if the process did not require steam, which necessitates high energy input and a large water recycle. It would also be desirable if the process did not require oxygen as a co-feed, so as to avoid the combination of oxygen with hydrocarbons and formation of combustion products. It would be more desirable if the process achieved acceptable conversion of alkyl aromatic compound, high selectivity to vinyl aromatic compound, and low selectivities to cracking and oxidation by-products. It would be even more desirable if the dehydrogenation catalyst was capable of simultaneously dehydrogenating mixtures of an alkyl aromatic compound and an alkane, such as ethylbenzene and ethane, to form product mixtures containing vinyl aromatic compound and an alkene, such as styrene and ethylene. Potentially, such a process might be applicable to an integrated process of producing styrene from a raw materials base comprised of benzene and ethane.

### SUMMARY OF THE INVENTION

In one aspect, this invention provides for a novel process of dehydrogenating an alkyl aromatic compound to form a vinyl aromatic compound. The novel process comprises contacting a dehydrogenation feedstream comprising an alkyl aromatic compound with a dehydrogenation catalyst comprised of gallium and zinc deposited on a catalyst support, the contacting being conducted under reaction conditions sufficient to produce a dehydrogenation product stream comprising the vinyl aromatic compound.

In a related aspect of this invention, the dehydrogenation feedstream may additionally comprise an alkane, and the dehydrogenation product stream may additionally comprise an alkene.

The novel dehydrogenation process of this invention finds utility in the preparation of vinyl aromatic compounds of industrial significance, including styrene, p-methylstyrene, α-methylstyrene, and divinylbenzene. Moreover, if an alkane is present in the dehydrogenation feedstream, then both vinyl aromatic compound and alkene can be produced simultaneously. Advantageously, the process of this invention does not employ steam. Accordingly, the process of this invention eliminates the need for water recycle and may consume less energy than steam-based processes. Secondly, the process of this invention does not employ oxygen. Accordingly, safety problems associated with handling mixtures of hydrocarbons and oxygen are also eliminated. Additionally, by avoiding the use of oxygen, the loss of raw material to combustion by-products is essentially eliminated.

Most advantageously, the process of this invention achieves acceptable conversion of alkyl aromatic compound and high selectivity to vinyl aromatic compound, as compared with prior art processes. All of the aforementioned advantages render the dehydrogenation process of this invention an improvement over the prior art.

In a second aspect, this invention provides for a novel integrated process of preparing a vinyl aromatic compound using as a raw material base an aromatic compound and an alkane. In this aspect the process comprises (a) contacting an alkane with a first dehydrogenation catalyst under reaction conditions sufficient to produce an alkene; (b) contacting the alkene with an aromatic compound in the presence of an alkylation catalyst under reaction conditions sufficient to produce an alkyl aromatic compound; and (c) contacting the alkyl aromatic compound with a second dehydrogenation catalyst, comprised of gallium and zinc deposited on a catalyst support, the contacting being conducted under reaction conditions sufficient to produce the vinyl aromatic compound.

In a related aspect of the aforementioned integrated process, the gallium-zinc catalyst employed in step (c) is also employed in step (a). In another related aspect of this invention, dehydrogenation steps (a) and (c) are conducted simultaneously in the same reactor with the same gallium-zinc dehydrogenation catalyst.

The integrated process described hereinabove can be employed to provide vinyl aromatic compound, such as styrene, from a raw material base comprised of aromatic compound, such as benzene, and alkane, such as ethane. In contrast, prior art processes traditionally produce the vinyl aromatic compound from a raw material base comprised of aromatic compound and alkene, the latter being derived from large, complex, and capital-intensive cracker units. The integrated process of this invention beneficially allows for the production of vinyl aromatic compound without the need for a capital-intensive cracker. Moreover, when the dehydrogenation of alkyl aromatic compound and alkane are conducted simultaneously in one reactor, the entire integrated process to form vinyl aromatic compound beneficially requires only one dehydrogenation unit and one alkylation unit.

In a third aspect, this invention pertains to a catalyst composition comprising gallium and zinc deposited on a transitional alumina support. The aforementioned catalyst composition is suitably employed in dehydrogenation processes, including the dehydrogenation of light paraffins and alkyl aromatic compounds to olefins and vinyl aromatic compounds, respectively.

### DETAILED DESCRIPTION OF THE INVENTION

The invention described herein involves, in one aspect, a novel process of dehydrogenating an alkyl aromatic compound, such as ethylbenzene, to form a vinyl aromatic compound, such as styrene. Beneficially, the process of this invention can, be integrated into a larger process of preparing a vinyl aromatic compound, such as styrene, from a raw material base comprising an aromatic compound, such as benzene, and an alkane, such as ethane. Features of the integrated process will become apparent to those of skill in the art as the individual aspects of this invention are fully described hereinbelow.

In its first aspect, the novel process comprises contacting a dehydrogenation feedstream comprising an alkyl aromatic compound with a dehydrogenation catalyst under reaction conditions sufficient to produce a dehydrogenation product stream comprising the vinyl aromatic compound. The catalyst employed in the process of this invention comprises gallium and zinc deposited on a catalyst support.

In a preferred embodiment, the dehydrogenation process is conducted in the absence of oxygen. The phrase “absence of oxygen” means that oxygen is not fed to the reactor as a co-reactant. Trace amounts of oxygen, however, may be present in the reactor, inasmuch as the process may be preferably conducted at sub-atmospheric pressures, and as such, the total exclusion of oxygen may be difficult to implement.

In another preferred embodiment of the dehydrogenation process of this invention, the alkyl aromatic compound is ethylbenzene or isopropylbenzene, and the vinyl aromatic compound is styrene or α-methylstyrene.

In a related aspect, the dehydrogenation feedstream additionally comprises an alkane, preferably, ethane. Under such circumstances, the dehydrogenation product stream additionally comprises an alkene, preferably, ethylene.

In a second aspect, this invention provides for a novel integrated process of preparing a vinyl aromatic compound using as a raw material base an alkyl aromatic compound and an alkane. In this aspect the process comprises (a) contacting an alkane with a first dehydrogenation catalyst under reaction conditions sufficient to produce an alkene; (b) contacting the alkene with an aromatic compound in the presence of an alkylation catalyst under reaction conditions sufficient to produce an alkyl aromatic compound; and (c) contacting the alkyl aromatic compound with a second dehydrogenation catalyst, comprised of gallium and zinc deposited on a catalyst support, the contacting being conducted under reaction conditions sufficient to produce the vinyl aromatic compound. In a preferred aspect of this invention, the gallium-zinc catalyst used in step (c) is also employed in step (a) to dehydrogenate the alkane. In another preferred embodiment, steps (a) and (c) are conducted simultaneously in the same reactor unit using the same gallium-zinc catalyst, for example, by feeding the reactor output from step (b) comprising the alkyl aromatic compound, with the alkane feed, to the reactor of step (a).

The novel process of simultaneously dehydrogenating alkyl aromatic compound and alkane is beneficially suited for integrated processes that convert a raw material base of aromatic compound and alkane to vinyl aromatic compound. The process is preferably suitable for converting a raw material base comprised of ethane and benzene or substituted benzene to styrene or substituted styrene. In a preferred embodiment, therefore, the novel integrated process comprises (a) contacting ethane with a first dehydrogenation catalyst under reaction conditions sufficient to produce ethylene; (b) contacting ethylene with benzene or a substituted benzene in the presence of an alkylation catalyst under reaction conditions sufficient to produce ethylbenzene or a substituted ethylbenzene; and (c) contacting ethylbenzene or the substituted ethylbenzene with a second dehydrogenation catalyst under reaction conditions sufficient to produce styrene or a substituted styrene; wherein the second dehydrogenation catalyst used in step (c) comprises gallium and zinc deposited on an alumina support. In another preferred embodiment, steps (a) and (c) for the dehydrogenation of ethane and ethylbenzene or substituted ethylbenzene are conducted simultaneously in the same reactor unit using the aforementioned gallium-zinc catalyst.

In a most preferred embodiment, this invention comprises an integrated process of preparing styrene comprising (a) feeding ethane to a dehydrogenation reactor wherein the ethane is dehydrogenated in the presence of a dehydrogenation catalyst under reaction conditions sufficient to prepare ethylene; (b) feeding the ethylene to an alkylation reactor wherein the ethylene is contacted with benzene in the presence of an alkylation catalyst under reaction conditions sufficient to prepare ethylbenzene; and (c) feeding the ethylbenzene to the dehydrogenation reactor of step (a) wherein the ethylbenzene is dehydrogenated with the dehydrogenation catalyst under reaction conditions sufficient to prepare styrene; the dehydrogenation catalyst comprising gallium and zinc deposited on an alumina support.

In a preferred embodiment of the aforementioned inventions, the dehydrogenation catalyst support comprises a transitional alumina support, as described hereinafter.

In yet another preferred embodiment, the dehydrogenation catalyst composition has a surface area of greater than about 20 m²/g and less than about 280 m²/g.

In a third aspect, this invention provides for a novel catalyst composition useful in the above-identified dehydrogenation processes. The novel catalyst composition comprises gallium and zinc deposited on a transitional alumina support.

Any alkyl aromatic compound can be employed in the dehydrogenation process of this invention, provided that a vinyl aromatic compound is produced. The aromatic moiety of the vinyl aromatic compound can comprise, for example, a monocyclic aromatic ring, such as benzene; a fused aromatic ring system, such as naphthalene; or an aromatic ring assembly, such as biphenyl. Preferably, the aromatic moiety is a monocyclic aromatic ring, more preferably, benzene. The alkyl segment of the alkyl aromatic compound can comprise any saturated, straight or branched chain hydrocarbon radical, or cyclic hydrocarbon radical, provided that the alkyl radical can be dehydrogenated to a vinyl radical. Non-limiting examples of suitable alkyl radicals include ethyl, n-propyl, iso-propyl, n-butyl, iso-butyl, t-butyl, cyclopentyl, cyclohexyl, and higher homologues thereof. Preferably, the alkyl radical is a C₂-C₁₀ alkyl radical, more preferably, a C₂-C₅ alkyl radical, and most preferably, ethyl or isopropyl. Optionally, the alkyl aromatic compound may be substituted on the aromatic ring with one or more substituents, in addition to the alkyl radical; or the alkyl radical itself may be substituted with one or two substituents. The substituents may be active or inactive towards dehydrogenation; but preferably should not interfere with the desired dehydrogenation process. Suitable substituents include, for example, alkyl moieties, such as methyl, hydroxy, ether, keto, and acid moieties. Non-limiting examples of alkyl aromatic compounds that may be beneficially employed in the process of this invention include ethylbenzene, isopropylbenzene, t-butyl-ethylbenzene, ethyltoluene, ethylxylene, ethylnaphthalene, ethylbiphenyl, isopropylnaphthalene, isopropylbiphenyl, diethylbenzene, and the like. Preferably, the alkyl aromatic compound is a C₈-C₃₀ alkyl aromatic compound, more preferably, a C₈-C₁₅ alkyl aromatic compound, and most preferably, ethylbenzene or a substituted derivative thereof (herein to include ethyltoluene, ethylxylene, diethylbenzene, and α-methylethylbenzene, i.e., isopropylbenzene).

As an optional reactant, the dehydrogenation feed may also contain an alkane, which for this invention shall be defined as any saturated aliphatic hydrocarbon having two or more carbon atoms that is capable of being dehydrogenated to an alkene (olefin). The alkane may be straight-chained, branched, or cyclic. The alkane may be substituted at any carbon with one or more substituents, provided that such substituents do not substantially interfere with the dehydrogenation of the alkyl aromatic compound or the alkane itself. Suitable non-limiting examples of alkanes include ethane, propane, butane, pentane, hexane, heptane, and octane, including straight and branched isomers thereof and higher homologues thereof; as well as cyclopentane and cyclohexane; and any mixtures of the foregoing compounds. Preferably, the alkane is a C₂₋₁₀ alkane, more preferably, a C₂₋₆ alkane, most preferably, ethane or propane.

Optionally, a diluent may be provided with the alkyl aromatic feedstream. The diluent functions to dilute the reactants and products for improved selectivity. Alternatively, the diluent may aid in the transfer and equilibration of heat or shift the equilibrium towards the desired products. Any gas that is substantially inert with respect to the dehydrogenation process may be suitably employed as the diluent including, for example, nitrogen, argon, helium, carbon dioxide, methane, and mixtures thereof. The concentration of diluent in the alkyl aromatic feedstream can vary depending, for example, upon the specific diluent, alkyl aromatic compound, catalyst, and dehydrogenation conditions selected. Typically, the concentration of diluent is greater than about 20 volume percent, preferably, greater than about 40 volume percent, and more preferably, greater than about 70 volume percent, based on the total volume of the dehydrogenation feedstream, including alkyl aromatic compound, diluent, and optional feed, such as alkane. Typically, the concentration of diluent is less than about 98 volume percent, preferably, less than about 90 volume percent, based on the total volume of the dehydrogenation feedstream.

Oxygen is not required for the dehydrogenation process of this invention. Preferably, the process is conducted in the absence of oxygen, which shall be taken to mean that oxygen is not fed to the reactor as a co-reactant. Likewise, steam is not required for the dehydrogenation process of this invention, and preferably, is not fed to the reactor. For clarification, it is noted that air or oxygen is typically employed in a separate regenerator to bum off coke on the dehydrogenation catalyst, and carbon dioxide is produced during such regeneration.

The catalyst employed in the dehydrogenation of the alkyl aromatic compound beneficially comprises gallium and zinc deposited on a catalyst support. The gallium and zinc loadings may be any appropriate loadings such that the catalyst functions to dehydrogenate an alkyl aromatic compound and optionally an alkane to yield a vinyl aromatic compound and optionally an alkene. Typically, the gallium loading is greater than about 0.1 percent, preferably, greater than about 0.5 percent, by weight, calculated as gallium oxide (Ga₂O₃) and based on the total weight of the catalyst. Typically, the gallium loading is less than about 10.0 percent, and preferably, less than about 4.0 percent, by weight, calculated as gallium oxide (Ga₂O₃) and based on the total weight of the catalyst. Typically, the zinc loading is greater than about 0.01 percent, and preferably, greater than about 0.10 percent, by weight, calculated as zinc oxide (ZnO) and based on the total weight of the catalyst. Typically, the zinc loading is less than about 8.0 percent, and preferably, less than about 0.8 percent, calculated as zinc oxide (ZnO) and based on the total weight of the catalyst.

Optionally, the dehydrogenation catalyst comprising gallium and zinc deposited on a catalyst support may additionally comprise one or more promoters selected from the group consisting of Group IA and Group IIA elements of the Periodic Table, and mixtures thereof. The Group IA and IIA promoter element(s) may function to increase catalyst activity, or increase selectivity to the desired dehydrogenation product, or increase catalyst lifetime, or provide a combination of such positive effects. Preferred Group IA elements include lithium, sodium, potassium, rubidium, and cesium, more preferably, potassium. Preferred Group IIA elements include magnesium, calcium, strontium, and barium. When one or more Group IA and/or Group IIA promoters are employed, then the total quantity of such elements is typically greater than about 0.01 weight percent, and preferably, greater than about 0.1 weight percent, calculated as oxide and based on the total weight of the catalyst composition. If one or more Group IA and/or Group IIA promoters are employed, then the total quantity thereof is typically less than about 5 weight percent, and preferably, less than about 1 weight percent, calculated as oxide and based on the total weight of the catalyst composition.

Optionally, the catalyst of this invention may contain one or more platinum group metal(s), which function to promote combustion during the regeneration of the catalyst with air or oxygen. The platinum group metals include ruthenium, rhodium, palladium, osmium, iridium, platinum, and any mixture thereof. Preferably, the platinum group metal is platinum. Typically, the minimum amount of platinum group metal is used to avoid unnecessary increases in catalyst cost. Typically, the loading of platinum group metal is greater than about 1 part per million (ppm) by weight, based on the total weight of the catalyst composition. Typically, the loading of platinum group metal is less than about 100 ppm by weight, preferably, less than about 50 ppm by weight, based on the total weight of the catalyst composition.

Optionally, the catalyst of this invention may also contain manganese, which also functions to improve combustion during regeneration of the catalyst under air or an oxygen-containing gas. If manganese is used, then the loading of manganese is typically greater than about 0.01 percent by weight, calculated as elemental manganese and based on the total weight of the catalyst composition. Typically, the loading of manganese is less than about 3 percent, and preferably, less than about 1 percent, by weight.

The catalyst support can be any conventional support that functions as a carrier for the active catalytic elements and optional promoters and other additives, so long as the carrier does not inhibit the dehydrogenation process of this invention. Suitable supports include, without limitation, alumina, silica, silica-aluminas, aluminosilicates, zirconia, titania, and the like. Preferably, the support comprises alumina, more preferably a transitional alumina, suitable examples of which include gamma, delta, theta, and eta aluminas, and mixtures thereof. Mixtures of the aforementioned transitional aluminas with alpha alumina are also suitable. More preferably, the alumina comprises a delta or theta transitional alumina or mixture thereof, optionally combined with alpha alumina. Mixtures of alumina with other support materials, such as silica, in any suitable combination, may also be employed. If silica is present, then preferably, the quantity of silica ranges from greater than about 0.5 to less than about 10 percent, by weight, based on the total weight of the support.

The dehydrogenation catalyst of this invention comprising gallium and zinc deposited on a catalyst support can be prepared by conventional methods, including for example impregnation, deposition precipitation, and ion-exchange. Preferably, the catalyst is prepared by impregnation, which is known in the art and described, for example, by Charles N. Satterfield in Heterogeneous Catalysis in Practice, McGraw-Hill Book Company, New York, 1980, 82-84. A more preferred preparation involves impregnation to incipient wetness, wherein an impregnation solution is wetted onto the support to the point of incipient wetness. One or more impregnating solutions may be employed, if desired.

Soluble gallium compounds and salts that may be suitably employed in the impregnation solution include, for example, gallium nitrate, gallium halides, gallium carboxylates, and other such soluble salts of gallium. Soluble zinc compounds and salts that may be suitably employed in the impregnation solution include, for example, zinc nitrate, zinc halides, zinc bicarbonate, and zinc carboxylates. Similar salts and compounds of manganese may be employed. Likewise, the noble metal can be impregnated from solutions of soluble salts or organometallic complexes. The incorporation of promoter elements into the catalyst composition may be effected in an analogous manner.

The impregnation solution(s) may be prepared with aqueous or non-aqueous solvents; although typically water is preferred. The concentration of selected soluble compound or salt in the impregnation solution typically ranges from about 0.01 M to the solubility limit. The impregnation may be conducted at any convenient temperature and pressure. Generally, the impregnation temperature is greater than about 10° C. and less than about 100° C., more preferably, about ambient temperature, taken as about 22° C. The impregnation is typically conducted at ambient pressure, but other pressures may also be suitable. Following impregnation with the desired elements, the support is calcined at a temperature sufficient to yield the dehydrogenation catalyst of the invention. Generally, the calcination temperature is greater than about 400° C., preferably, greater than about 500° C. Generally, the calcination temperature is less than about 1,100° C., and preferably, less than about 850° C.

In a preferred preparation, zinc is deposited onto the support in an initial impregnation, and then the support is calcined at a temperature between about 850° C. and 1000° C. After calcination, the remaining catalytic elements, including gallium, optional promoters, manganese, and noble metal, are impregnated onto the support from one or more solutions. Thereafter, the fully impregnated support is calcined at a temperature between about 700° C. and 850° C. This preferred method wherein the support is pretreated with zinc in an initial step prior to deposition of gallium and other elements beneficially improves ethylbenzene conversion and selectivities to desired products.

At the current time, the preferred catalyst species comprising gallium and zinc deposited on alumina does not exhibit a gallium-zinc-aluminum mixed oxide of spinel structure or other distinct structure, as determined by X-ray analysis. The diffraction pattern, as presently understood, establishes alumina phases. On use of the catalyst in the dehydrogenation process disclosed herein, the gallium, zinc, and optional alkali promoter tend to migrate from the surface of the support to sub-surface regions, up to about 1 micron in depth, as determined by X-ray photoelectron spectroscopy.

In yet another embodiment, the aforementioned dehydrogenation catalyst composition can be bound, compacted, or extruded with or deposited onto a secondary support that may function, for example, to bind and strengthen the catalyst particles and improve attrition resistance. Non-limiting examples of suitable secondary supports include alumina, silica, silica-alumina, silicon carbide, titanium oxide, zirconium oxide, zirconium silicate, as well as other similar refractory oxides and ceramic supports, and combinations thereof. A preferred secondary support is silica. The quantity of secondary support that may be used can vary depending upon the specific catalyst components; but typically, the quantity of secondary support comprises greater than about 1 weight percent, based on the total weight of the catalyst composition and secondary support. Typically, the quantity of secondary support comprises less than about 30 weight percent, and preferably, less than about 20 weight percent, based on the total weight of the catalyst composition including secondary support. When silica is used, it may be preferable, to employ from greater than about 1 percent to less than about 5 percent silica.

The catalyst used for the dehydrogenation of alkane in step (a) of the integrated process, described herein, may be any catalyst that functions in such capacity, for example, as described in U.S. Pat. No. 5,196,634, U.S. Pat. No. 5,633,421, and EP-B1-0,637,578, incorporated herein by reference. In a preferred embodiment, the catalyst used in the dehydrogenation of alkane comprises gallium and zinc deposited on a catalyst support, the catalyst being identical to that employed in the dehydrogenation of alkyl aromatic compound.

Various conventional reactor designs are acceptable for the dehydrogenation of alkyl aromatic compound and alkane including fixed bed, transport bed, and fluidized bed reactors, operating under continuous flow or intermittent flow modes. A fluidized bed reactor is preferred. More preferably, the fluidized bed reactor contains internal structures (internals) that facilitate plug flow behavior. In a preferred embodiment, the reactor is designed for countercurrent flow, such that the dehydrogenation feed and the catalyst are fed at opposite ends of the reactor and flow in opposite directions. Preferably, the dehydrogenation catalyst is continuously transported out of the reactor to a regenerator for regeneration; after which the regenerated catalyst is recycled back to the dehydrogenation reactor.

No limitations need be placed on catalyst particle size, shape, or density, provided that the catalyst is suited for the selected reactor design and active in the dehydrogenation process. If the catalyst is provided to a fluidized bed reactor, as is the preferred mode of operation, then the catalyst average particle diameter, shape, and density should be such as to provide for acceptable attrition resistance and acceptable flow and transport properties. Preferably, the catalyst has the properties of, and is classified as, a Group-A particle according to Geldart (Gas Fluidization Technology, D. Geldart, John Wiley & Sons). Accordingly, the average catalyst particle diameter is typically greater than about 5 microns (μm), and preferably, greater than about 25 μm. Typically, the average catalyst particle diameter is less than about 500 μm, and preferably, less than about 150 μm. The surface area of the catalyst typically exceeds about 20 m²/g, as determined by the BET (Brunauer-Emmet-Teller) method, described by S. Brunauer, P. H. Emmett, and E. Teller, Journal of the American Chemical Society, 60, 309 (1938). Preferably, the surface area is greater than about 40 m²/g, more preferably, greater than about 60 m²/g, and most preferably, greater than about 80 m²/g. Typically, however, the surface area is less than about 280 m²/g, and more preferably, less than about 150 m²/g. Preferably, the catalyst particles are smooth with rounded edges, are substantially non-cohesive, and possess an attrition resistance appropriate for use in a fluidized bed reactor, as known to those of skill in the art.

When a fluidized bed reactor is employed for the dehydrogenation process, then optionally a sweeping gas may be used in the process of this invention. Fluidized bed reactors usually comprise at least two zones: a reaction zone for the fluidized bed and a freeboard zone above the fluidized bed. The freeboard zone comprises a free space that allows for expansion of the catalyst volume on fluidization. The sweeping gas is typically introduced into the freeboard zone and primarily functions to remove products from the freeboard zone so as to minimize undesirable thermal reactions. Any gas that is substantially inert with respect to the dehydrogenation process may be suitably employed as the sweeping gas, including, for example, nitrogen, argon, helium, carbon dioxide, and mixtures thereof. The concentration of sweeping gas in the freeboard zone can vary widely, depending, for example, upon the specific alkyl aromatic and/or alkane feeds and specific process conditions employed, particularly, temperature and gas velocity. Typically, the concentration of sweeping gas in the freeboard zone is greater than about 10 volume percent, and preferably, greater than about 20 volume percent. Typically, the concentration of sweeping gas in the freeboard zone is less than about 90 volume percent, and preferably, less than about 70 volume percent.

If desired, the dehydrogenation feedstream may be preheated before entry into the dehydrogenation reactor. Any preheat temperature can be used, provided it lies below the temperature at which thermal cracking of the alkyl aromatic and/or alkane becomes measurable. Typical preheat temperatures are greater than about 150° C., preferably, greater than about 250° C., and more preferably, greater than about 350° C. Typical preheat temperatures are less than about 500° C., and preferably, less than about 400° C.

The temperature of the dehydrogenation zone can be any operable temperature, provided that a vinyl aromatic compound and/or alkene are produced in the process. The operable dehydrogenation temperature will vary with the specific catalyst and reactant feed. Typically, the dehydrogenation temperature is greater than about 400° C., and preferably, greater than about 425° C. Typically, the dehydrogenation temperature is less than about 750° C. and, preferably, less than about 675° C. Below about 550° C., the conversions of alkyl aromatic compound and alkane may be too low; whereas above about 675° C., thermal cracking of the reactants may occur. In fluidized bed reactors, the temperature is typically measured on the catalyst bed in fluidized form.

The dehydrogenation process can be conducted at any operable total pressure, ranging from subatmospheric to superatmospheric, provided that the vinyl aromatic product is produced, and if desired, the alkene. If the total reactor pressure is too high, the equilibrium position of the dehydrogenation process may be shifted backwards towards alkyl aromatic compound and optionally alkane. Preferably, the process is conducted under vacuum to maximize the yield of vinyl aromatic product and optionally alkene. Preferably, the total pressure is greater than about 1 psia (6.9 kPa), more preferably, greater than about 3 psia (20.7 kPa). Preferably, the total pressure is less than about 73 psia (503.3 kPa), more preferably, less than about 44 psia (303.4 kPa). Most preferably, the total pressure is subatmospheric, ranging between about 3 psia (20.7 kPa) and about 13 psia (90.6 kPa). In a fluidized bed reactor, the pressure throughout the freeboard and reaction zones may vary depending upon process factors, such as the weight and buoyancy of the catalyst and frictional effects.

The gas hourly space velocity of the dehydrogenation reactant feedstream will depend upon the specific alkyl aromatic compound and catalyst employed, the specific vinyl aromatic product formed, the reaction zone dimensions (e.g., diameter and height), and the form and weight of the catalyst particles. For the dehydrogenation of alkane to alkene, analogous variations in space velocity are found. It is desirable to remove the reactant and products quickly from the reactor, so as to reduce thermal cracking and other undesirable side reactions. In fluidized bed reactors specifically, gas flow should be sufficient to induce fluidization of the catalyst bed. Generally, the space velocity of the dehydrogenation feedstream varies from the minimum velocity needed to achieve fluidization of the catalyst particles to a velocity just below the minimum velocity needed to achieve pneumatic transport of the catalyst particles. Fluidization occurs when the catalyst particles are disengaged, when the particles move in a fluid-like fashion, and when the bed pressure drop is essentially constant along the bed. Pneumatic transport occurs when an unacceptable quantity of catalyst particles is entrained in the gas flow and transported out of the reactor. Preferably, the space velocity of the dehydrogenation feedstream varies from the minimum bubbling velocity to a bubbling velocity just below the minimum turbulent flow velocity. Bubbling occurs when gas bubbles can be seen in the fluidized bed, but little back-mixing of gas and solids occurs. Turbulent flow occurs when both substantial bubbling and substantial back-mixing of gas and solids occur. More preferably, the flow is sufficient to cause bubbling, but not substantial back-mixing.

In view of the above, the normal gas hourly space velocity (GHSV), calculated as the total flow of dehydrogenation feedstream comprising alkyl aromatic compound, optional diluent, optional sweeping gas, and optional alkane is typically greater than about 60 ml total feed per ml catalyst per hour (h⁻¹), measured at standard conditions of atmospheric pressure and 0° C. Preferably, the GHSV of the dehydrogenation stream is greater than about 120 h⁻¹, and more preferably, greater than 300 h⁻¹ at standard conditions. Generally, the GHSV of the dehydrogenation stream is less than about 10,000 h⁻¹, preferably, less than about 3,600 h⁻¹, and more preferably, less than 700 h⁻¹, measured as total flow at standard conditions.

For this invention, the gas residence time in the dehydrogenation zone may be calculated as the height of the reaction zone times the reaction zone voidage fraction divided by the superficial gas velocity of the reaction feedstream. The “reaction zone voidage fraction” is the fraction of the reaction zone which is empty. The “superficial gas velocity” is the gas velocity through the empty reactor. Typically, the gas residence time in the reaction zone is greater than about 0.3 seconds (sec), measured at operating conditions. Preferably, the gas residence time in the reaction zone is greater than about 1 sec, more preferably, greater than about 2 sec, measured at operating conditions. Generally, the gas residence time in the reaction zone is less than about 60 sec, preferably, less than about 30 sec, and more preferably, less than about 5 sec, measured at operating conditions.

When an alkyl aromatic compound is contacted with the dehydrogenation catalyst in the manner described hereinbefore, a vinyl aromatic compound is produced. Ethylbenzene, for example, is converted primarily to styrene. Likewise, ethyltoluene is converted to p-methylstyrene (p-vinyltoluene); t-butylethylbenzene is converted to t-butylstyrene; isopropylbenzene (cumene) is converted to α-methylstyrene; and diethylbenzene is converted to divinylbenzene. Hydrogen is also formed during dehydrogenation. By-products produced in lower yields include benzene, toluene, tar, and coke.

The conversion of the alkyl aromatic compound in the process of this invention can vary depending upon the specific feed composition, catalyst, reactor, and process conditions used. For the purposes of this invention, the term “conversion of alkyl aromatic compound” is defined as the mole percentage of alkyl aromatic compound converted to all products. In this process, the conversion of alkyl aromatic compound is typically greater than about 30 mole percent, preferably, greater than about 40 mole percent, and more preferably, greater than about 50 mole percent.

Likewise, the selectivity to products will vary depending upon the specific feed composition, catalyst, reactor, and process conditions. In this context, “selectivity” is defined as the mole percentage of converted alkyl aromatic compound that forms a specific product, preferably, vinyl aromatic compound. In the process of this invention, the selectivity to vinyl aromatic compound, preferably styrene or substituted styrene, is typically greater than about 70 mole percent, preferably, greater than about 80 mole percent, and more preferably, greater than about 90 mole percent.

All of the aforementioned dehydrogenation process conditions may be employed as described or modified by those of skill in the art to facilitate the dehydrogenation of the alkane to alkene. Conventional alkane dehydrogenation catalysts may be used in step (a) of the integrated process; however, advantageously, the gallium-zinc catalyst described herein may be suitably employed for alkane dehydrogenation, preferably, simultaneously with alkyl aromatic dehydrogenation (step (c)). The conversion of alkane and selectivity to alkene achieved varies analogously as well. Typically, the alkane conversion, defined as the mole percentage of alkane converted to all products, is greater than about 30 mole percent, preferably, greater than about 40 mole percent, and more preferably, greater than about 50 mole percent. Typically, the selectivity to alkene, defined as the mole percentage of converted alkane that forms alkene, is greater than about 70 mole percent, preferably, greater than about 80 mole percent, and more preferably, greater than about 90 mole percent.

When the dehydrogenation catalyst is sufficiently deactivated, it may be transported to a separate zone for regeneration. Regeneration typically involves burning of coke on the catalyst and/or re-oxidizing active sites under air or oxygen, or some diluted variation thereof. The regeneration feedstream comprising deactivated catalyst, optional diluent and sweeping gas can be preheated prior to introduction into the regenerator. A typical preheat temperature is greater than about 200° C., preferably, greater than about 300° C., and more preferably, greater than about 400° C. The preheat temperature is typically less than about 650° C., and preferably, less than about 630° C. Typically, the regeneration temperature lies below the minimum temperature for thermally cracking the alkyl aromatic compound and vinyl aromatic product and any optional alkane and alkene. Accordingly, the regeneration temperature is typically greater than about 400° C., and preferably, greater than about 570° C. Typically, the regeneration temperature is less than about 850° C. and, preferably, less than about 775° C.

The gas hourly space velocity of regeneration gas comprising air, oxygen, or diluted variation thereof, through the regenerator can be broadly varied, provided that the catalyst is regenerated at least in part. Typically, the gas hourly space velocity (GHSV), calculated as the total of the regeneration gas, is greater than about 60 ml total feed per ml catalyst per hour (h⁻¹), and preferably, greater than about 100 h⁻¹, measured under standard conditions (0° C., 1 atm). Generally, the gas hourly space velocity of the regeneration gas is less than about 5,000 h⁻¹, preferably, less than about 1,000 h⁻¹, measured under standard conditions.

In the regeneration zone, the gas residence time, calculated as the height of the regeneration zone times the regeneration zone voidage fraction divided by the superficial gas velocity of the total of the regeneration gas is greater than about 0.3 sec, measured at operating conditions. The “regeneration zone voidage fraction” is the fraction of the regeneration zone which is empty. Preferably, the gas residence time in the regeneration zone is greater than about 1 sec, and more preferably, greater than about 5 sec. Generally, the gas residence time in the regeneration zone is less than about 60 sec, preferably, less than about 30 sec. and more preferably, less than about 10 sec, measured at operating conditions.

In the integrated process contemplated in this invention, an alkane is dehydrogenated to an alkene; thereafter, an aromatic compound is alkylated with the alkene to form an alkyl aromatic compound; and the alkyl aromatic compound is dehydrogenated to form a vinyl aromatic compound. As noted above, alkane dehydrogenation can be conducted using prior art process methods, or the methods disclosed in this invention. The alkylation step can be conducted with any conventional alkylation catalyst and process conditions known to those of skill in the art, as illustrated for example, in U.S. Pat. No. 5,430,211, U.S. Pat. No. 4,409412, U.S. Pat. No. 5,157,185, U.S. Pat. No. 4,107,224, U.S. Pat. No. 5,856,607, and EP-B1-0,432,814, incorporated herein by reference.

The invention will be further clarified by a consideration of the following examples, which are intended to be purely illustrative of the use of the invention. Other embodiments of the invention will be apparent to those skilled in the art from a consideration of this specification or practice of the invention as disclosed herein. All percentages are weight percent, unless otherwise noted.

Preparation of Catalyst Support

A microspheroidal alumina support was prepared by spray drying a mixture of hydrated alumina and Ludoxg silica (1.4+0.2 percent) and then heating the resulting spray dried particles at a temperature above 1000° C. sufficient to achieve a particle surface area of 70+10 m₂/g for Examples 1, 2 and CE-1. X-ray diffraction analysis of the alumina product detected alpha (23 percent), theta (21 percent), and delta phases (56 percent) as the major components. The alumina support was dried at 150° C. for a minimum of 12 hours prior to use. For Examples 3, 4, and CE-2, a more mild calcination procedure was used to obtain a higher surface area of 100+/−10 m²/g that contained less alpha alumina phase. The catalysts of the following examples and comparative experiments were prepared by incipient wetness techniques using aqueous solutions and the thusly-prepared transitional alumina support.

### EXAMPLE 1

A catalyst illustrative of the invention was prepared comprised of gallium oxide (Ga₂O₃, 1.7 percent), potassium oxide (K₂O, 0.6 percent), zinc oxide (ZnO, 0.1 percent), platinum (100 ppm), balance alumina support (70 m²/g). A solution containing gallium nitrate (0.0371 moles Ga), potassium nitrate (0.025 moles K), tetra-amine platinum(II) nitrate (0.09 millimole Pt), and zinc nitrate hexahydrate (0.80 g, 2,69 millimoles) in 64 ml of deionized water was prepared and added slowly with good mixing to the alumina (200 grams) at room temperature. A 6 ml aliquot of water was used to rinse the flask and then added to the support. The impregnated alumina was kept at room temperature for 2 h, then 80° C. for 2 h and next 150° C. for 12 h. The material was finally calcined at 750° C. for 4 h and cooled to room temperature to yield a sample of catalyst.

The catalyst was sieved between 100 and 400 mesh (0.149 mm to 0.037 mm) screens. A 91 ml sample of catalyst (1.17 g/ml packed, bulk density) was loaded into a 1 inch (2.5 cm) ID up-flow fluidized bed quartz reactor. A diluent gas was employed in the reaction, comprising ethane with a flow rate of 0.645 L/min (volume at 0° C., 1 atm (100 kPa) pressure). The reactor was heated to 550° C. as monitored and controlled from an internal thermocouple placed in the center of the reactor and two inches (5.50 cm) from the bottom of the catalyst bed. An ethylbenzene flow (0.68 ml/min) was initiated, mixed with the diluent, and vaporized at 195° C. prior to introduction to the reactor. The sum of ethane and ethylbenzene flows gave a gas hourly space velocity (GHSV) of 500 h⁻¹, based on the packed volume of the catalyst and ideal gas volumes at normal conditions (0° C., 1 atm pressure). One evaluation cycle consisted of a reaction segment and a regeneration segment, which were separated by nitrogen purges. The reaction segment lasted for 10 min. Next, a nitrogen purge was passed through the reactor for 15 min. The liquid products were condensed in a liquid nitrogen trap, and the residual gaseous products were captured in a gas sampling bag. The nitrogen feed was switched to air at the same flow rate and the reactor temperature was increased to 650° C. for a catalyst regeneration segment. The regeneration segment was maintained for 30 min and followed by a second nitrogen purge. The gas stream was collected in a separate bag during the regeneration portion of the cycle and analyzed by gas chromatography (gc). The liquid sample was weighed and analyzed by gc. The results were quantified using external standards and normalization. Compilation of the three analyses allowed the calculation of an overall conversion and selectivity for the entire cycle. The main products were styrene, benzene, toluene, alpha-methyl styrene, tar, and coke. Tar is defined as the sum of peaks eluted after alpha-methyl styrene to the end of the temperature ramp of 230° C. The molecular weight of stilbene was used for the average molecular weight of tar. Coke was measured as CO₂ formed during regeneration. The reactor was cooled to the next reaction temperature while purging with nitrogen. The temperature was set for the next cycle, and the entire process was repeated to generate another data point. Cycles were completed at selected temperatures between 550° C. and 600° C. The first six catalytic cycles were used as a break-in period, and the data were not recorded. The catalyst performance was stable through 200 cycles. The resulting data are shown in Table 1.

TABLE 1
Dehydrogenation of Ethylbenzene (With Zinc)^{1,2}

| | EB | Sty | Ben | Tol | AMS | Tar | Coke |
|---|---|---|---|---|---|---|---|
| T | Conv | Sel | Sel | Sel | Sel | Sel | Sel |
| ° C. | Mol % | Mol % | Mol % | Mol % | Mol % | Mol % | Mol % |
| 550 | 41.6 | 94.0 | 1.8 | 0.9 | 0.1 | 1.8 | 1.4 |
| 552 | 42.7 | 95.6 | 1.6 | 0.8 | 0.0 | 0.6 | 1.3 |
| 578 | 48.8 | 91.4 | 2.6 | 1.7 | 0.1 | 2.7 | 1.5 |
| 574 | 48.9 | 94.1 | 2.8 | 1.5 | 0.1 | 0.1 | 1.4 |
| 575 | 49.1 | 93.1 | 2.8 | 1.6 | 0.1 | 1.2 | 1.2 |
| 577 | 49.4 | 92.1 | 2.6 | 1.7 | 0.1 | 2.2 | 1.3 |
| 575 | 49.8 | 92.9 | 2.9 | 1.6 | 0.1 | 1.0 | 1.6 |
| 577 | 50.3 | 91.0 | 3.0 | 1.8 | 0.1 | 2.0 | 2.1 |
| 600 | 52.7 | 88.2 | 5.2 | 3.2 | 0.2 | 1.4 | 1.7 |
| 589 | 52.8 | 89.2 | 3.8 | 2.4 | 0.1 | 2.3 | 2.1 |
| 600 | 53.5 | 89.0 | 5.4 | 3.2 | 0.2 | 0.7 | 1.6 |
| 599 | 53.7 | 87.4 | 5.2 | 3.4 | 0.2 | 1.5 | 2.2 |

¹Reaction Conditions: Catalyst, Ga₂O₃, 1.7 percent; K₂O, 0.6 percent; ZnO, 0.1 percent; platinum, 100 ppm; balance alumina; atmospheric pressure; GHSV, 500 h⁻¹ (ethane plus ethylbenzene); ethylbenzene, 15 vol % gas feed.
²EB = ethylbenzene, Sty = styrene, Ben = benzene, Tol = toluene, AMS = alpha methyl styrene.

From Table 1 it is seen that the gallium-zinc catalyst achieved an ethylbenzene conversion ranging from roughly 42 to 54 mole percent, and a styrene selectivity ranging from 91.0 to 95.6 mole percent. Cracking by-products, tar and coke were produced at acceptably low levels. Table 2 illustrates the molar ratio of ethylene to styrene obtained in the dehydrogenation output stream.

TABLE 2
Molar Ratio of Ethylene (ET) to Styrene (STY) in
Dehydrogenation Effluents

| | | |
|---|---|---|
| | | Comparative |
| Example 1 | Example 2 | Experiment 1 |

| T (° C.) | ET/STY | T (° C.) | ET/STY | T (° C.) | ET/STY |
|---|---|---|---|---|---|
| 551.8 | 0.929 | 550.3 | 0.832 | 576.1 | 0.984 |
| 574.4 | 1.067 | 550.4 | 0.835 | 576.7 | 1.038 |
| 574.8 | 1.056 | 575.6 | 0.866 | 577.3 | 0.899 |
| 575.2 | 1.125 | 576.1 | 0.972 | 577.6 | 0.936 |
| 576.6 | 1.127 | 576.3 | 0.937 | 586.7 | 1.014 |
| 576.8 | 0.993 | 576.8 | 0.887 | 591.5 | 1.150 |
| 577.7 | 0.991 | 594.4 | 1.044 | 600.4 | 1.123 |
| 599.0 | 1.263 | 598.3 | 0.084 | 600.7 | 1.195 |
| 599.6 | 1.188 | 599.6 | 1.061 | 600.8 | 1.262 |
| 600.3 | 1.147 | — | — | — | — |

Table 2 shows that nearly equal amounts of ethylene and styrene were produced under the dehydrogenation conditions illustrated in Example 1. Such a product ratio of ethylene and styrene can provide a useful industrial stream for integrated styrene and ethylene systems.

### EXAMPLE 2

A second embodiment of the catalyst was prepared comprised of gallium oxide (Ga₂O₃, 1.7 percent), potassium oxide (K₂O, 0.6 percent), zinc oxide (ZnO, 0.5 percent), and platinum (100 ppm), balance alumina support (70 m²/g) prepared hereinabove. The catalyst was prepared in the manner described in Example 1, with the exception that 3.8 g (12.6 millimole) of zinc nitrate hexahydrate were added in the impregnating solution. The catalyst was evaluated in the dehydrogenation of ethylbenzene in a manner closely similar to that described in Example 1, with the following changes. The catalyst, 87 ml (packed) having a density of 1.21 g/packed ml, was loaded into the reactor. The ethane flow was 0.616 liters/min, and the liquid ethylbenzene flow was 0.65 ml/min. Results are shown in Table 3.

TABLE 3
Dehydrogenation of Ethylbenzene (With Zinc)^{1,2}

| | EB | Sty | Ben | Tol | AMS | Tar | Coke |
|---|---|---|---|---|---|---|---|
| T | Conv | Sel | Sel | Sel | Sel | Sel | Sel |
| ° C. | Mol % | Mol % | Mol % | Mol % | Mol % | Mol % | Mol % |
| 550 | 41.4 | 96.0 | 1.0 | 0.5 | 0.0 | 0.3 | 2.2 |
| 550 | 42.1 | 94.2 | 1.4 | 0.6 | 0.1 | 2.1 | 1.6 |
| 576 | 43.6 | 95.6 | 1.2 | 0.8 | 0.1 | 0.9 | 1.4 |
| 576 | 44.7 | 95.3 | 1.6 | 0.9 | 0.1 | 0.8 | 1.3 |
| 576 | 44.9 | 93.9 | 1.2 | 0.8 | 0.1 | 1.6 | 2.3 |
| 577 | 45.0 | 94.8 | 1.3 | 0.9 | 0.1 | 1.6 | 1.4 |
| 594 | 49.9 | 94.6 | 2.1 | 1.5 | 0.1 | 1.6 | 0.0 |
| 598 | 51.0 | 94.6 | 2.1 | 1.5 | 0.1 | 1.6 | 0.0 |
| 600 | 52.1 | 91.3 | 2.4 | 1.7 | 0.1 | 2.4 | 2.1 |

¹Reaction Conditions: Catalyst, Ga₂O₃, 1.7 percent; K₂O, 0.6 percent; ZnO, 0.5 percent; platinum, 100 ppm; balance alumina; atmospheric pressure; GHSV, 500 h⁻¹ (ethane plus ethylbenzene); ethylbenzene, 15 vol % gas feed.
²EB = ethylbenzene, Sty = styrene, Ben = benzene, Tol = toluene, AMS = alpha methyl styrene.

From Table 3 it is seen that the catalyst comprising gallium, zinc, potassium, and platinum achieved an ethylbenzene conversion between about 41 and 52 mole percent and a styrene selectivity between about 91 and 96 mole percent, with acceptably low selectivities to cracked by-products, tar, and coke. The ethylene to styrene mole ratio achieved in this example was close to 1/1, as shown in Table 2.

### Comparative Experiment CE-1

A comparative catalyst was prepared comprised of gallium oxide (Ga₂O₃, 1.7 percent), potassium oxide (K₂O, 0.6 percent), and platinum oxide (PtO, 100 ppm) on the alumina (70 m²/g) described hereinabove. The preparation was similar to that of Example 1, with the exception that no zinc nitrate hexahydrate was impregnated onto the support. The comparative catalyst was evaluated in the dehydrogenation of ethylbenzene according to the procedure of Example 1, with the following changes. A catalyst sample of 389 ml (packed) and a density of 1.19 g/packed ml was loaded into the reactor. The ethane flow was 0.630 liters/min, and the liquid ethylbenzene flow was 0.67 ml/min. The results are shown in Tables 2 and 4.

TABLE 4
Dehydrogenation of Ethylbenzene (Without Zinc)^{1,2}

| | EB | Sty | Ben | Tol | AMS | Tar | Coke |
|---|---|---|---|---|---|---|---|
| T | Conv | Sel | Sel | Sel | Sel | Sel | Sel |
| ° C. | Mol % | Mol % | Mol % | Mol % | Mol % | Mol % | Mol % |
| 576 | 45.3 | 91.8 | 3.5 | 2.0 | 0.1 | 1.2 | 1.4 |
| 578 | 47.5 | 91.8 | 3.4 | 2.1 | 0.1 | 0.8 | 1.7 |
| 577 | 45.8 | 91.9 | 3.7 | 2.3 | 0.2 | 0.1 | 1.9 |
| 577 | 47.8 | 91.1 | 3.9 | 2.2 | 0.1 | 1.0 | 1.8 |
| 587 | 50.0 | 89.8 | 4.6 | 2.7 | 0.2 | 1.4 | 1.3 |
| 592 | 51.3 | 87.8 | 5.3 | 3.1 | 0.2 | 1.4 | 2.2 |
| 590 | 52.0 | 88.3 | 5.3 | 3.3 | 0.2 | 1.2 | 1.7 |
| 600 | 52.3 | 86.4 | 7.2 | 4.3 | 0.3 | 0.1 | 1.6 |
| 601 | 53.5 | 84.5 | 7.3 | 4.2 | 0.3 | 1.7 | 2.0 |
| 601 | 54.7 | 84.6 | 6.8 | 4.0 | 0.3 | 1.9 | 2.4 |

¹Reaction Conditions: Catalyst, Ga₂O₃, 1.7 percent; K₂O, 0.6 percent; platinum, 100 ppm; balance alumina; atmospheric pressure; GHSV, 500 h⁻¹ (ethane plus ethylbenzene); ethylbenzene, 15 vol % gas feed.
²EB = ethylbenzene, Sty = styrene, Ben = benzene, Tol = toluene, AMS = alpha methyl styrene.

From Table 2 it is seen that the molar ratio of ethylene to styrene in the product stream was again close to 1/1. When the data is Table 4 (Comparative Experiment 1) are compared with the data in Tables 1 and 3 (Examples 1 and 2), however, it is seen that at any given conversion, the zinc-promoted catalyst displayed a higher selectivity to styrene, as compared with the comparative catalyst not containing zinc. Accordingly, the addition of zinc to the gallium catalyst is advantageous.

### EXAMPLE 3

Another embodiment of the catalyst was prepared comprised of gallium oxide (Ga₂O₃, 2.0 percent), potassium oxide (K₂O, 0.6 percent), zinc oxide (ZnO, 0.5 percent), and manganese (0.17 percent), balance alumina support (100 m²/g) prepared hereinabove. The catalyst was prepared in the manner described in Example 1, with the exception that 3.6 g (12.1 millimole) of zinc nitrate hexahydrate were added in an initial impregnating solution. The resulting solid was dried in the manner of the previously described incipient wetness methods, then calcined at 950° C. for 6 hours. A second impregnation followed to add the gallium, potassium and manganese, and the resulting solid was subsequently dried and calcined at 750° C. for 4 hours. The catalyst was evaluated in the dehydrogenation of ethylbenzene in a manner closely similar to that described in Example 1, with the following changes. A 20% ethylbenzene/80% ethane reagent stream was used. The catalyst, 90 ml (packed) having a density of 1.02 g/packed ml, was loaded into the reactor. The ethane flow was 0.653 liters/min, and the liquid ethylbenzene flow was 0.90 ml/min. Results are shown in Tables 5 and 6.

TABLE 5
Dehydrogenation of Ethylbenzene (With Zinc)^{1,2}

| | EB | Sty | Ben | Tol | AMS | Tar | Coke |
|---|---|---|---|---|---|---|---|
| T | Conv | Sel | Sel | Sel | Sel | Sel | Sel |
| ° C. | Mol % | Mol % | Mol % | Mol % | Mol % | Mol % | Mol % |
| 575.4 | 45.1 | 92.3 | 2.8 | 1.2 | 0.1 | 0.2 | 3.4 |
| 594.4 | 49.6 | 89.3 | 3.8 | 2.0 | 0.1 | 0.4 | 4.4 |
| 609.3 | 51.4 | 86.6 | 4.4 | 2.9 | 0.2 | 0.1 | 5.9 |

¹Reaction Conditions: Catalyst, Ga₂O₃, 2.0 percent; K₂O, 0.6 percent; ZnO, 0.5 percent; manganese, 0.17%; balance alumina support; atmospheric pressure; GHSV, 500 h⁻¹ (ethane plus ethylbenzene); ethylbenzene, 20 vol % gas feed.
²EB = ethylbenzene, Sty = styrene, Ben = benzene, Tol = toluene, AMS = alpha methyl styrene.

TABLE 6
Molar Ratio of Ethylene (ET) to Styrene (STY)
in Dehydrogenation Effluents

| | | |
|---|---|---|
| | | Comparative |
| Example 3 | Example 4 | Experiment 2 |

| | | | | | |
|---|---|---|---|---|---|
| T (° C.) | ET/STY | T (° C.) | ET/STY | T (° C.) | ET/STY |

| | | | | | |
|---|---|---|---|---|---|
| 575.4 | 0.659 | 576.8 | 0.599 | 573.6 | 0.567 |
| 594.4 | 0.786 | 594.9 | 0.651 | 593.8 | 0.694 |
| 609.3 | 0.834 | 611.0 | 0.671 | 607.3 | 0.758 |

From Table 5 it is seen that the catalyst comprising gallium, zinc, potassium, and manganese achieved an ethylbenzene conversion between about 45 and 51 mole percent and a styrene selectivity between about 87 and 92 mole percent, with acceptably low selectivities to cracked by-products, tar, and coke. Moreover, the zinc pre-treatment produced a significant catalytic improvement in activity and selectivity with respect to an identical catalyst with no zinc pre-treatment, as illustrated in Comparative Experiment 2 (CE-2) hereinbelow. From Table 6 it is seen that the catalyst comprising gallium and zinc also dehydrogenated ethane to ethylene in an ethylene:styrene ratio of between about 0.66:1 to 0.83:1.

### EXAMPLE 4

Another embodiment of the catalyst was prepared comprised of gallium oxide (Ga₂O₃, 2.0 percent), potassium oxide (K₂O, 0.6 percent), zinc oxide (ZnO, 5.0 percent), and manganese (0.17 percent), balance alumina support (100 m²/g) prepared hereinabove. The catalyst was prepared in the manner described in Example 1, with the exception that 38.5 g (129 millimole) of zinc nitrate hexahydrate were added in an initial impregnating solution. The resulting solid was dried in the manner of the previously described incipient wetness methods, then calcined at 950° C. for 6 hours. A second impregnation followed to add the gallium, potassium and manganese, and the resulting solid was subsequently dried and calcined at 750° C. for 4 hours. The catalyst was evaluated in the dehydrogenation of ethylbenzene in a manner closely similar to that described in Example 1, with the following changes. A 20% ethylbenzene/80% ethane reagent stream was used. The catalyst, 90 ml (packed) having a density of 1.07 g/packed ml, was loaded into the reactor. The ethane flow was 0.653 liters/min, and the liquid ethylbenzene flow was 0.90 ml/min. Results are shown in Tables 6 and 7.

TABLE 7
Dehydrogenation of Ethylbenzene (With Zinc)^{1,2}

| | EB | Sty | Ben | Tol | AMS | Tar | Coke |
|---|---|---|---|---|---|---|---|
| T | Conv | Sel | Sel | Sel | Sel | Sel | Sel |
| ° C. | Mol % | Mol % | Mol % | Mol % | Mol % | Mol % | Mol % |
| 576.8 | 42.2 | 93.6 | 2.0 | 1.2 | 0.1 | 0.5 | 2.5 |
| 594.9 | 44.9 | 92.4 | 2.4 | 1.8 | 0.1 | 0.6 | 2.7 |
| 611.0 | 46.1 | 89.4 | 3.0 | 2.6 | 0.1 | 0.8 | 4.0 |

¹Reaction Conditions: Catalyst, Ga₂O₃, 2.0 percent; K₂O, 0.6 percent; ZnO, 5.0 percent; manganese, 0.17 percent; balance alumina support; atmospheric pressure; GHSV, 500 h⁻¹ (ethane plus ethylbenzene); ethylbenzene, 20 vol % gas feed.
²EB = ethylbenzene, Sty = styrene, Ben = benzene, Tol = toluene, AMS = alpha methyl styrene.

From Table 7 it is seen that the catalyst comprising gallium, zinc, potassium, and manganese achieved an ethylbenzene conversion between about 42 and 46 mole percent and a styrene selectivity between about 89 and 94 mole percent, with acceptably low selectivities to cracked by-products, tar, and coke. Moreover, the zinc pre-treatment produced a significant catalyst improvement in selectivity with respect to an identical catalyst with no zinc pre-treatment, as shown in Comparative Experiment 2 (CE-2) hereinafter. From Table 6 it is seen that the catalyst comprising gallium and zinc also dehydrogenated ethane to ethylene in an ethylene:styrene ratio of between about 0.60:1 to 0.67: 1.

### Comparative Experiment (CE-2)

A comparative catalyst was prepared comprised of gallium oxide (Ga₂O₃, 2.0 percent), potassium oxide (K₂O, 0.6 percent), and manganese (Mn, 0.17 percent) on the alumina (100 m²/g) described hereinabove. The preparation was similar to that of Example 1, with the exception that no zinc nitrate hexahydrate was impregnated onto the support. The comparative catalyst was evaluated in the dehydrogenation of ethylbenzene according to the procedure of Example 1, with the following changes. A catalyst sample of 89 ml (packed) and a density of 1.02 g/packed ml was loaded into the reactor. The ethane flow was 0.645 liters/min, and the liquid ethylbenzene flow was 0.89 ml/min. The results are shown in Tables 6 and 8.

TABLE 8
Ethylbenzene Dehydrogenation (Without Zinc)^{1,2}

| | EB | Sty | Ben | Tol | AMS | Tar | Coke |
|---|---|---|---|---|---|---|---|
| T | Conv | Sel | Sel | Sel | Sel | Sel | Sel |
| ° C. | Mol % | Mol % | Mol % | Mol % | Mol % | Mol % | Mol % |
| 573.6 | 42.5 | 90.8 | 3.7 | 1.4 | 0.1 | 0.3 | 3.7 |
| 593.8 | 47.5 | 86.6 | 5.1 | 2.4 | 0.1 | 0.4 | 5.4 |
| 607.3 | 50.9 | 83.5 | 5.9 | 3.3 | 0.2 | 0.2 | 6.9 |

¹Reaction Conditions: Catalyst, Ga₂O₃, 2.0 percent; K₂O, 0.6 percent; manganese, 0.17 percent; balance alumina support; atmospheric pressure; GHSV, 500 h^{-1 (ethane plus ethylbenzene); ethylbenzene, 20 vol % gas feed.}
²EB = ethylbenzene, Sty = styrene, Ben = benzene, Tol = toluene, AMS = alpha methyl styrene.

From Table 6 it is seen that the catalyst comprising gallium without zinc dehydrogenated ethane to ethylene in an ethylene:styrene ratio about comparable to that achieved in Example 4, but somewhat lower than achieved in Example 3, with catalysts comprising gallium and zinc. When the data in Table 8 (CE-2) are compared with the data in Tables 5 and 7 (Examples 3 and 4), it is seen that at any given conversion, the zinc-promoted catalyst of the invention displayed a higher selectivity to styrene, as compared with the comparative catalyst not containing zinc. Accordingly, the addition of zinc to the gallium catalyst is advantageous.

1. A process of preparing a vinyl aromatic compound comprising contacting a dehydrogenation feedstream comprising an alkyl aromatic compound and optionally a diluent with a dehydrogenation catalyst under reaction conditions sufficient to prepare a dehydrogenation product stream comprising a vinyl aromatic compound; the catalyst comprising gallium and zinc deposited on a catalyst support.

2. The process of claim 1 wherein the alkyl aromatic compound is a C₈₋₃₀ alkyl aromatic compound.

3. The process of claim 1 wherein the alkyl aromatic compound is selected from ethylbenzene, ethyltoluene, ethylxylene, diethylbenzene, isopropylbenzene, and mixtures thereof.

4. The process of claim 1 wherein gallium is loaded on the catalyst support in an amount greater than about 0.1 and less than about 10 weight percent, calculated as is gallium oxide and based on the total weight of the catalyst composition.

5. The process of claim 1 wherein zinc is loaded on the catalyst support in an amount greater than about 0.01 and less than about 8 weight percent, calculated as zinc oxide and based on the total weight of the catalyst composition.

6. The process of claim 1 wherein the catalyst support is selected from alumina, silica, silica-alumina, aluminosilicates, titania, zirconia, and mixtures thereof.

7. The process of claim 6 wherein the catalyst support comprises a transitional alumina.

8. The process of claim 1 wherein the catalyst further comprises a promoter selected from elements of Group IA, Group IIA, and combinations thereof.

9. The process of claim 8 wherein the loading of promoter is greater than about 0.01 and less than about 5 weight percent, calculated as promoter metal oxide and based on the total weight of the catalyst composition.

10. The process of claim 1 wherein the catalyst further comprises manganese in a concentration from greater than about 0.01 to less than about 3 weight percent, calculated as elemental manganese and based on the total weight of the catalyst composition.

11. The process of claim 1 wherein the catalyst further comprises from greater than 1 ppm to less than about 100 ppm platinum group metal.

12. The process of claim 1 wherein the catalyst has a surface area of greater than about 20 m²/g and less than about 280 m²/g, and optionally, an average particle diameter of greater than about 5 microns and less about 500 microns.

13. The process of claim 1 wherein the catalyst composition is prepared by first depositing zinc onto the support and thereafter depositing gallium and optionally a Group IA or Group IIA element, a platinum group metal, manganese, or a mixture thereof onto the support.

14. The process of claim 1 wherein the temperature ranges from greater than about 400° C. to less than about 750° C.

15. The process of claim 1 wherein the pressure ranges from greater than about 1 psia (6.9 kPa) to less than about 73 psia (503.3 kPa).

16. The process of claim 1 wherein a diluent is used, and the diluent is selected from nitrogen, argon, helium, methane, carbon dioxide, and mixtures thereof.

17. The process of claim 1 wherein the process is conducted in a fluidized bed reactor.

18. The process of claim 1 wherein the dehydrogenation reaction is conducted in the absence of co-fed oxygen or similar oxidant and/or steam.

19. The process of claim 1 wherein the catalyst is transported to a regenerator for regeneration under air or oxygen at a temperature greater than about 400° C. and less than about 850° C.

20. The process of claim 1 wherein the vinyl aromatic compound comprises styrene, vinyltoluene, vinylxylene, t-butylstyrene, αmethylstyrene, divinylbenzene, or a mixture thereof.

21. The process of claim 1 wherein the dehydrogenation feedstream comprises an alkane, and the dehydrogenation product stream comprises an alkene.

22. The process of claim 1 wherein the dehydrogenation feedstream comprises ethylbenzene and ethane, and the dehydrogenation product stream comprises styrene and ethylene.

23. An integrated process of preparing a vinyl aromatic compound comprising (a) dehydrogenating an alkane in the presence of a first dehydrogenation catalyst under reaction conditions sufficient to prepare an alkene; (b) contacting the alkene with an aromatic compound in the presence of an alkylation catalyst under reaction conditions sufficient to prepare an alkyl aromatic compound; and (c) dehydrogenating the alkyl aromatic compound with a second dehydrogenation catalyst under reaction conditions is sufficient to prepare a vinyl aromatic compound; the catalyst for step (c) comprising gallium and zinc deposited on a catalyst support.

24. The process of claim 23 wherein the alkane is ethane; the alkene is ethylene; the aromatic compound is benzene; the alkyl aromatic compound is ethylbenzene; and the vinyl aromatic compound is styrene.

25. The process of claim 23 wherein the catalyst further comprises one or more elements selected from the group consisting of Group IA, Group IIA, manganese, and platinum group metals.

26. The process of claim 23 wherein the alkane dehydrogenation step (a) occurs simultaneously in the same reactor and with the same gallium-zinc catalyst as the alkyl aromatic dehydrogenation step (c).

27. The process of claim 23 wherein the catalyst support is a transitional alumina.

28. The process of claim 23 wherein the catalyst is prepared by first depositing zinc onto the support and thereafter depositing gallium and optionally a Group IA or Group IIA element, a platinum group metal, manganese, or a mixture thereof onto the support.

29. A catalyst composition comprising gallium and zinc, optionally manganese, optionally a platinum group metal, and optionally one or more Group IA or IIA metals, deposited on a transitional alumina support.

30. The catalyst composition of claim 29 wherein the catalyst is prepared by first depositing zinc onto the support and thereafter depositing gallium and any other optional elements onto the support.
